(19) United States
(12) Patent Application Publication
(10) Pub. No.: US 2022/0243257 A1
(43) Pub. Date: Aug. 4, 2022

(54) SYSTEM AND METHOD FOR GENERATING A PERSONALIZED PREDICTED PROTEOME

(71) Applicant: Nathaniel D. Kwok, Cupertino, CA (US)

(72) Inventor: Nathaniel D. Kwok, Cupertino, CA (US)

(21) Appl. No.: 17/590,601

(22) Filed: Feb. 1, 2022

Related U.S. Application Data
(60) Provisional application No. 63/144,122, filed on Feb. 1, 2021.

(51) Int. Cl. C12Q 1/6809; C12Q 1/6837

(52) U.S. Cl. CPC C12Q 1/6809; C12Q 1/6837

(57) ABSTRACT

A process for predicting a proteome based on one or more tissue samples of an individual may include: (a) identifying somatic and germline variants based on a reference genome and nucleotide sequences derived from the tissue samples; (b) constructing a customized genome by modifying the reference genome based on the somatic and germline variants identified; (c) aligning RNA sequences derived from the tissue samples to the customized genome; (d) assembling a detected transcriptome with transcripts derived from the aligned RNA sequences; and € associating the detected transcriptome with proteins in a protein database and including the associated proteins in the proteome. The tissue samples includes a tissue sample obtained from a diseased site (“target sample”) and a matched normal or virtual normal tissue sample.

### CROSS REFERENCE TO RELATED APPLICATIONS

The present application is related and claims priority of U.S. provisional application (“Provisional Application”), 63/144,122, entitled “SYSTEM AND METHOD FOR GENERATING A PERSONALIZED PREDICTED PROTEOME,” filed on Feb. 1, 2021. The disclosure of the Provisional Application is hereby incorporated by reference in their entirety.

### BACKGROUND OF THE INVENTION

### 1. Field of the Invention

The present invention relates to bioinformatics. In particular, the present invention relates to applying bioinformatics techniques to predict amino acid sequences that may be detected in tissue samples collected from an individual, based on genomic and transcriptomic data derived from both a reference genome and nucleotide sequences of the tissue samples.

### 2. Discussion of the Related Art

At the present time, identification of non-standard (“non-canonical”) or specimen-specific protein sequences via protein mass spectrometry methods is limited by a paucity of methods for generating complete and individualized protein sequence databases, comprising the specimen's own genetic code, that can serve as the necessary search space to supply to proteomics analysis algorithms. The article, “ProteomeGenerator: A Framework for Comprehensive Proteomics Based on de Novo Transcriptome Assembly and High-Accuracy Peptide Mass Spectral Matching” (“Cifani”), by P. Cifani et al., published in J. Proteome Res., 2018 Nov. 2, vol. 17(11), pp. 3681-3692, discloses a system for constructing such a predicted database. Using RNA sequencing data from patient tissue as input, Cifani's system (i) assembles an individualized set of potentially expressed gene transcripts (defined by chromosomal coordinates of exon boundaries) via de novo transcriptome assembly, (ii) converts loci to nucleotide sequences by reading out from a reference genome, and then (iii) outputs high likelihood translated (i.e. protein) reading frames as determined by an algorithmic scoring function. Such protein sequences form Cifani's predicted proteome, including all their permutational isoforms (“proteoforms”). As previously mentioned, this patient-specific proteome may then be used to guide mass spectrometric detection of peptides in the patient's tissue samples. In this manner, the method allows for discovery of peptides derived from de novo or non-canonical transcripts, such as those resulting from erroneous messenger RNA splicing.

Recently, the article “Spritz: A Proteomic Database Engine” (“Cesnik”), by A. J. Cesnik et al., in Journal of Proteome Research, on Sep. 23, 2020, at https://pubs.acs.org/action/showCitFormats?doi=10.1021/acs.jproteome.0c00407&ref=pdf, discloses augmenting a transcriptome that is derived, based on RNA sequence data from tissue samples and the Ensembl reference genome, using predictions from sequence variations and post-translation modifications. To predict post-translation modifications, Cesnik discloses using MetaMorpheus, a global post-translation discovery tool.

In addition to enabling detection and discovery of non-canonical amino acid sequences, the predicted proteome significantly facilitates one to trace back or map detected amino acid sequences found by mass spectrometry to genes in the reference genome. This tracing or mapping process is an essential step in many applications in precision medicine, such as finding targets that allow creation of personalized therapies. However, the process of transcribing a gene from the genome to a messenger RNA (mRNA) in a cell involves complex manipulation. For example, the cell may splice various coding portions (“exons”) of the genomic sequence together, while excluding non-coding portions (“introns”) of the genomic sequence). Many of the splicing processes have not been properly annotated in the reference genomes or are yet unknown. Mutations (e.g., substitution, insertion, or deletion of nucleotides, gene fusion or any combination thereof) further complicate such tracing back or mapping of the transcriptome back to reference genome. In some cases, where the mutation significantly changes the exome (e.g., gene fusion), it may not be possible or meaningful to relate the transcripts back to the reference genome. Furthermore, in certain precision medicine applications where, to identify therapeutic targets, it is necessary not only to map sample-specific proteome to variants from the reference genome, but also mapping the sample-specific proteome to variants specific to the individual's genome.

A tool optimized for predicting sample-specific amino acid sequences in an individual's tissue samples from specific sequencing or structural variants in the individual's own genome is highly desirable.

### SUMMARY

According to one embodiment of the present invention, a process for predicting a proteome based on one or more tissue samples of an individual includes: (a) identifying somatic and germline variants based on a reference genome and nucleotide sequences derived from the tissue samples; (b) constructing a customized genome by modifying the reference genome based on the somatic and germline variants identified; (c) aligning RNA sequences derived from the tissue samples to transcription loci in the customized genome; (d) assembling a detected transcriptome with transcripts derived from the aligned RNA sequences; and (e) associating the detected transcriptome with proteins in a protein database and including the associated proteins in the proteome. The tissue samples may include both a tissue sample obtained from a diseased site (“target sample”) and (optionally) a matched normal or virtual normal tissue sample.

In this context, somatic variants may include, relative to the alleles in the matched normal or virtual normal tissue sample, alternative alleles found in the target sample. Likewise, germline variants may include, relative to the alleles in the reference genome, alternative alleles found in either the target sample or the matched normal or virtual normal sample.

In one embodiment, the nucleotide sequences used in a process of the present invention may be provided from a whole genome sequencing (WGS) or whole exome sequencing (WES) procedure.

In some embodiments, the somatic and germline variants may include structural rearrangement variants other than single-nucleotide polymorphisms and short insertion or deletion mutations. The germline variants identified may be assessed for quality using a deep-learning model, which may be implemented on a convolutional neural network, or any suitable machine learning techniques.

In one embodiment, the process may detect in the aligned RNA sequences transcripts that correspond to structural rearrangements in the customized genome. The structural rearrangements may include gene fusion, tandem or exon duplications, or combinations thereof. The detected transcripts that correspond to structural rearrangements are then included to augment the assembled detected transcriptome. The process may further include extracting exons from transcripts in the assembled detected transcriptome and using the extracted exons to identify open read frames in the customized genome. The open reading frames may further facilitate identification of proteins in the protein database.

According to another embodiment of the present invention, a bioinformatics system configurable and operable on one or more processors, optionally including one or more neural networks, may include: (a) a variant calling module configured to identify somatic and germline variants based on a reference genome and nucleotide sequences derived from the tissue samples, and simultaneously extensible to accept as input arbitrary genomic variant input specifiable in a variant call file (VCF); (b) a customized genome module configurable to construct a customized genome based on modifying the reference genome according to the variants identified and supplied (if any); and (c) a customized transcriptome assembly module configurable to: (i) align RNA sequences derived from the tissue samples to transcription loci in the customized genome; (ii) assemble a detected transcriptome with transcripts derived from the aligned RNA sequences; and (iii) translate the detected transcriptome into predicted protein sequences comprising the individualized proteome. The one or more processors and any neural networks may be accessible by a user of the bioinformatics system over a wide area computer network (WAN) or otherwise specified computational cluster.

The present invention is better understood upon consideration of the detailed description below, in conjunction with the accompanying drawings.

### BRIEF DESCRIPTION OF THE DRAWINGS

FIG. 1 is a flow diagram illustrating system 20 for assembling a predicted customized proteome, in accordance with one embodiment of the present invention.

FIG. 2 illustrates the operations of alignment and preprocessing module 200, according to one embodiment of the present invention.

FIG. 3 illustrates the operations of variant calling module 300, according to one embodiment of the present invention.

FIG. 4 illustrates the operations of customized diploid genome module 300, according to one embodiment of the present invention.

FIG. 5, which includes FIG. 5A and FIG. 5B and the figure key, illustrates the operations of customized transcriptome assembly module 400, according to one embodiment of the present invention.

FIG. 6 illustrates the operations of gene fusion module 500, according to one embodiment of the present invention.

FIG. 7 illustrates the operations of variant transcript expansion module 600, according to one embodiment of the present invention.

### DETAILED DESCRIPTION OF THE PREFERRED EMBODIMENTS

According to one embodiment of the present invention, a system and a method assemble a predicted proteome based on (i) DNA sequences of a customized genome constructed using tissue samples from an individual (e.g., a patient), and (ii) RNA sequences from the tissue samples. In this detailed description, the term “customized genome” refers to a genome incorporating germline and somatic variants identified from the tissue samples. The terms “sample-specific genome” and “patient-specific genome” may each be used in the detailed description interchangeably with the term “customized genome.” The term “customized transcriptome” refers to a transcriptome incorporating germline and somatic variants in the customized genome. The term “customized predicted proteome” refers to a predicted proteome derived from a customized genome and a customized transcriptome. The term “customized predicted proteomic database” refers to a database containing amino acid sequences derived from the customized predicted proteome.”

In one embodiment, the present invention implements in a computer or computer system a “pipeline” for assembling a customized predicted proteome based on a customized genome. In this context, a pipeline refers to an application of a specific set of tools—often software or customized, application-specific hardware—in a specific sequence (“workflow”) on a data set. Each tool in the pipeline typically performs a specific function, accepting input data conforming to a specific set of requirements, and providing output data conforming to the specific set of requirements for input into the next tool in the pipeline. In some embodiments, the workflow may be defined in one or more user-editable script files. In this regard, the pipeline may be entered at multiple entry points, so long as, at each entry point, the requirements on the input data at that entry point are satisfied. The pipeline may also be exited at any of a number of exit points at the user's specification. Because of the complexity of a pipeline in bioinformatics applications, the workflow may be controlled using a pipeline tool, e.g., snakemake. Some embodiments of the present invention may be implemented using open-source tools. In this detailed description, many examples are illustrated using tools from the Picard, GATK, bcftools SAMtools and Transdecoder toolkits that are known to those of ordinary skill in the art.

In some embodiments, the pipeline executes on a computer system accessible by a user over a wide area computer network (i.e., “cloud” implementations). A suitable computer system for a pipeline of the present invention may include a processor cluster that is optimized for high-performance computation-bound, statistical or machine-learning operations. The computer system may include machine-learning modules (e.g., neural networks implemented by, for example, parallel arithmetic or graphic processors and embedded memory circuits). In one embodiment, a Linux-based operating system controls the operations of the processor cluster.

### Overview

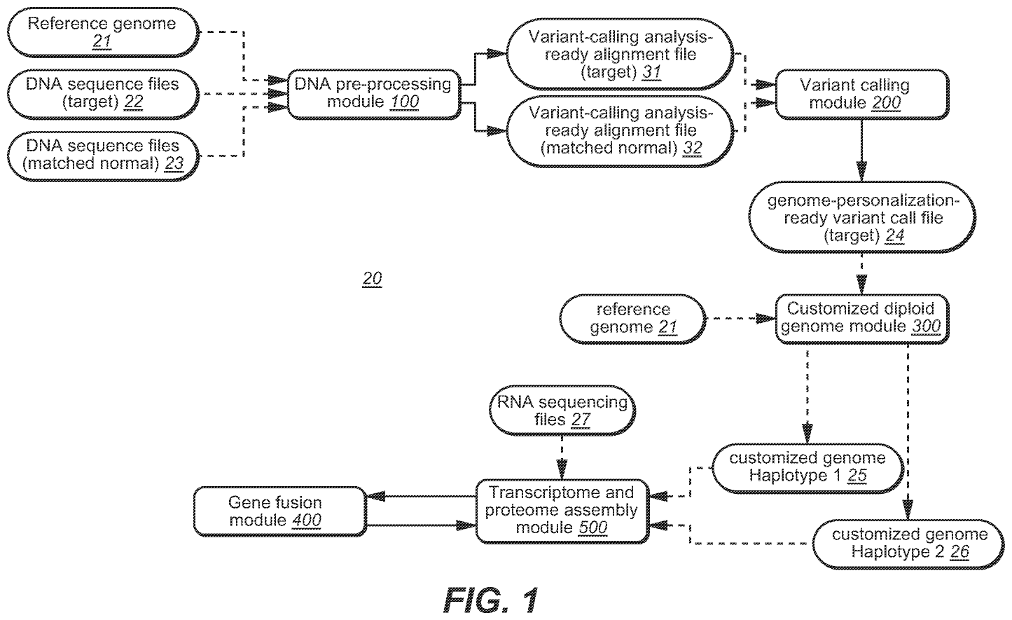

FIG. 1 is a flow diagram illustrating system 20 for assembling a customized predicted proteome based on a customized genome, in accordance with one embodiment of the present invention. As shown in FIG. 1, system 20 includes: (a) alignment and pre-processing module 100, (b) variant calling module 200, (c) customized diploid genome module 300, (d) gene fusion module 400, and (d) customized transcriptome assembly module 500.

As shown in FIG. 1, alignment and pre-processing module 100 receives as input (i) reference genome 21 (e.g., human genome assembly GRCH38, maintained by the Genome Reference Consortium), (ii) optional matched normal (MN) DNA sequence file or files 23, which includes DNA sequencing data obtained from a tissue sample of an individual or a close relative of the individual, and (iii) target DNA sequence file or files 22, which includes DNA sequencing data obtained from a target tissue sample of the individual. The MN tissue sample is used as a reference (e.g., a believed healthy tissue sample, in the context of a pathology application). The predicted customized proteome is intended for facilitating the detection of the proteins in the targeted tissue sample, as well as in MN tissue sample, if desired for comparative analysis. In this context, nucleotide sequences of DNA or RNA fragments are colloquially referred in the art as “reads.” From these input files, alignment and pre-processing module 100 provides output files 31 and 32 suitable for use in calling somatic and germline variants, which may include: (i) alternative alleles found in target DNA sequence file or files 22, relative to MN DNA sequence file or files 23 (“somatic alternative alleles”), or (ii) alternative alleles found in target DNA sequence file or files 22, or MN DNA sequence file or files 23, relative to reference genome 21 (“germline alternative alleles”).

Variant calling and annotation module 200 takes output files 31 and 32 of assignment and pre-processing modules 100 to produce a called variants file 24 in a standardized format (e.g., variant call format (VCF)). Alternatively, a VCF containing an arbitrary set of variants can be supplied as input (file 24) in lieu of the called variants generated via the preceding steps.

Using the annotated called variants file 24 from variant calling and annotation module 200, customized diploid genome module 300 creates the customized genome in file sets 25 and 26, containing (i) homozygous germline alternative alleles and the consensus alleles in the reference genome (“first Haplotype”), and (ii) the germline alternative alleles and the somatic alternative alleles (“second Haplotype”), respectively. Note that the term “Haplotype” in this detailed description does not refer to alleles inherited from the same parent. The term “Haplotype” herein is used to distinguish alleles included in file sets 25 and 26 of files in the customized genome.

File sets 25 and 26 (“first Haplotype files” and “second Haplotype files,” respectively) are provided to customized transcriptome assembly module 400, which also receives as input RNA sequencing file 27. Customized transcriptome assembly module 400 aligns and indexes the reads in RNA sequencing file 27 separately to the first and second Haplotypes in the customized genome. Based on the alignment, a transcriptome is assembled for each Haplotype using scaffolds of overlapping read sequences. In some instances, where the variant callers in variant caller 200 do not handle more complicated mutations (e.g., chromosomal rearrangements, such as gene fusion), an additional transcript extraction tool (e.g., gene fusion module 500) may be used to augment the transcriptome of each Haplotype, when it is desired to detect and to extract transcripts containing the more complicated mutations that may be present in the customized genome. The transcriptomes of both Haplotypes allow for extraction of nucleotide sequences which are translated in 6 reference frames to amino acid sequences, and a Markov model-based scoring function selects the most probable protein-coding reading frames. Each of the resulting Haplotype-wise predicted proteomes are subsequently merged to provide the customized predicted proteome.

Gene fusion module 500 is an optional module that augments the transcripts in the transcriptome assembled by customized transcriptome assembly module 400. In some embodiments, the germline and somatic variants that drive customized transcriptome assembly module 400 encompass only single-nucleotide polymorphisms (SNPs), indels (i.e., insertion or deletion), and certain multi-nucleotide polymorphisms (MNPs). Gene fusion module 500 detects and assembles in the customized genome more complicated mutations (e.g., chromosomal rearrangements).

Variant transcript expansion module 600 is an optional module that addresses the situation where variants exist in close chromosomal proximity to one another, such that a short peptide fragment (e.g., a 5-30 amino acid tryptic peptide analyzed often in proteomics) is likely to span multiple variant loci. Since it is not yet facile to resolve with sufficient accuracy the relative physical nucleic acid strands on which each variant resides, it is advantageous to account for all 2^{N} possible nucleotide fragment combinations, where N is the number of variants spanning the given fragment, in the subsequent transcript and protein libraries. In that regard, the variant transcript expansion module compiles these fragment combinations and merges them with the libraries generated from transcriptome assembly module 400 and gene fusion module 500.

### Alignment and Pre-Processing Module 100

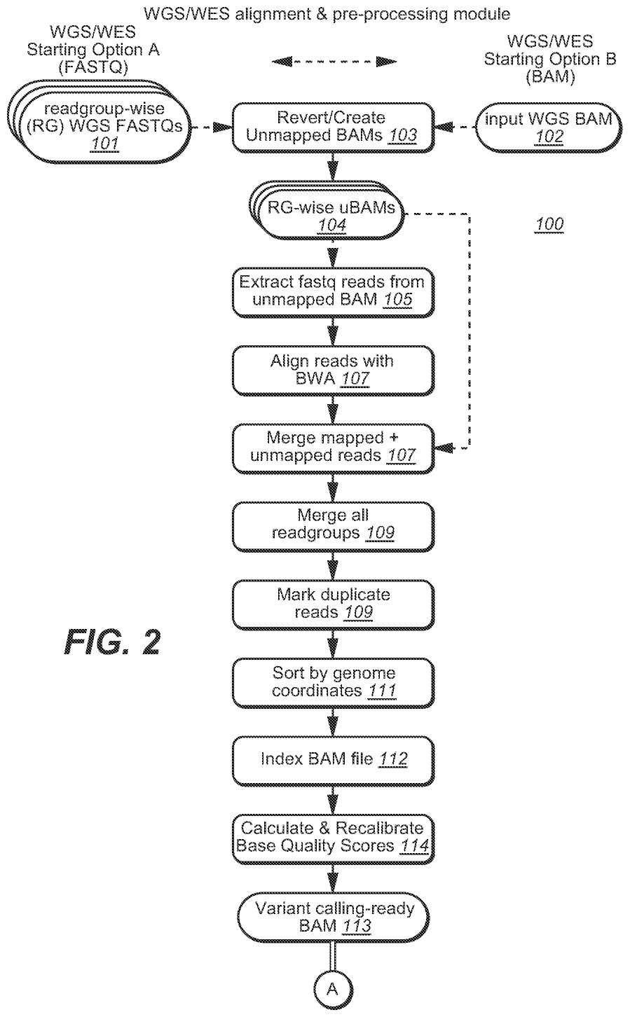

FIG. 2 illustrates the operations of alignment and preprocessing module 100, according to one embodiment of the present invention. In one embodiment, alignment and preprocessing module 100 receives as input one or more whole genome sequencing (WGS) files or one or more whole exome sequencing (WES) files, each representing the result of WGS or WES DNA sequencing of a tissue sample obtained from an individual. (In the application illustrated by FIG. 1, alignment and preprocessing module 100 is run twice: once using, as the WES or WGS input files, matched normal (MN) DNA sequence file or files 23, and once using, as the WES or WGS input files, target DNA sequence file or files 22.)

As illustrated in FIG. 2, the WGS or WES files may be in FASTQ format (e.g., FASTQ files 101), which is often the output file format of a commercial sequencing instrument. Alternatively, the WGS or WES files may be in a sequence alignment map (SAM) format or a binary sequence alignment map (BAM) format (e.g., SAM or BAM files 102). These input files each contain nucleotide sequences of DNA fragments or “reads”, typically having a mean length between 50-400 base pairs. The reads may already be aligned or “mapped” to a reference genome, or they may be unmapped. The reads may also be results from single or multiple sequencing instrument runs. It is customary to collectively refer to the reads of a single sequencing instrument run as belonging to the same “read group,” although this usage is not universally adhered to. The reads may also be “paired-end” or “single-end” reads, depending on the sequencing protocol, as known to those of ordinary skill in the art.

As it is customary for bioinformatics tool to include results of their operations as text annotations that mark-up their input files, some reads in the input files to alignment and preprocessing module 100 may already include alignment information from previous processing. To avoid inconsistencies, it is generally good practice to un-map the input files, as indicated at step 103. One suitable tool for un-mapping, for example, is the RevertSAM program in Picard. Un-mapped files 104 result upon completion of un-mapping step 103.

In this implementation, alignment is performed using the Burrow-Wheeler aligner BWM-MEM, which prefers input files in the FASTQ format and provides BAM format output files. Accordingly, at step 105, any SAM or BAM input files are converted to FASTQ format (e.g., FASTQ files 106) using, for example, the SAMtoFASTQ tool in Picard. At step 107, alignment is performed relative to a user-specified reference genome (e.g., human genome assembly GRCH38 (e.g., reference genome 21 of FIG. 1), maintained by the Genome Reference Consortium). At step 107, the reads in the output files of tool BWM-MEM may be sorted by names for efficiency. This sorting can be achieved using, for example, the Sort tool in SAMtools. Unlike many bioinformatics applications, this embodiment of the present invention does not discard or set aside reads that cannot be aligned (“unmapped reads”). Rather, the unmapped reads are included with the mapped reads. If the alignment tool discards unmapped reads, the unmapped reads can be recovered from un-mapped files 104. Merging the unmapped and the mapped reads may be achieved using, for example, the MergeBAMAlignment tool in Picard. Where a read may be mapped to more than one location (i.e., the “primary alignment”), the additional mapped locations (“secondary alignments”) are also kept. Merged files 108, in BAM format, result.

If the reads in BAM files 108 originate from multiple read groups, step 109 merge the read groups, placing the reads into a single file (e.g., merged file 110). Merging step 109 may be achieved using, for example, the Merge tool in SAMtools. The reads in merged file 110 may be sorted at step 111 according to their genomic coordinates using the Sort tool in SAMtools. A genomic coordinate for a nucleotide may be, for example, a chromosome number and a position in the chromosome. An index from genomic coordinates to mapped reads may then be compiled in step 112 using, for example, the Index tool in SAMtools. The quality scores associated with the reads are then recalculated and renormalized (i.e., “Base Quality Score Recalibration (BQSR)”) at step 114 using, for example, the BaseRecalibrator tool and the ApplyBQSR tool in GATK. The resulting recalibrated BAM files 113 are then ready for variant calling (as indicated in FIG. 1 by circle “A”).

In the application illustrated by FIG. 1, BAM files 113 that result from running alignment and preprocessing module 100 on MN or VN DNA sequence file or files 23 are suitable for use in calling germline variants (i.e., BAM files 113 for that run correspond to output file 32). Likewise, BAM files 113 that result from running alignment and preprocessing module 100 on target DNA sequence file or files 23 are suitable for use in calling somatic variants (i.e., BAM files 113 for that run correspond to output file 31).

### Variant Calling Module 200

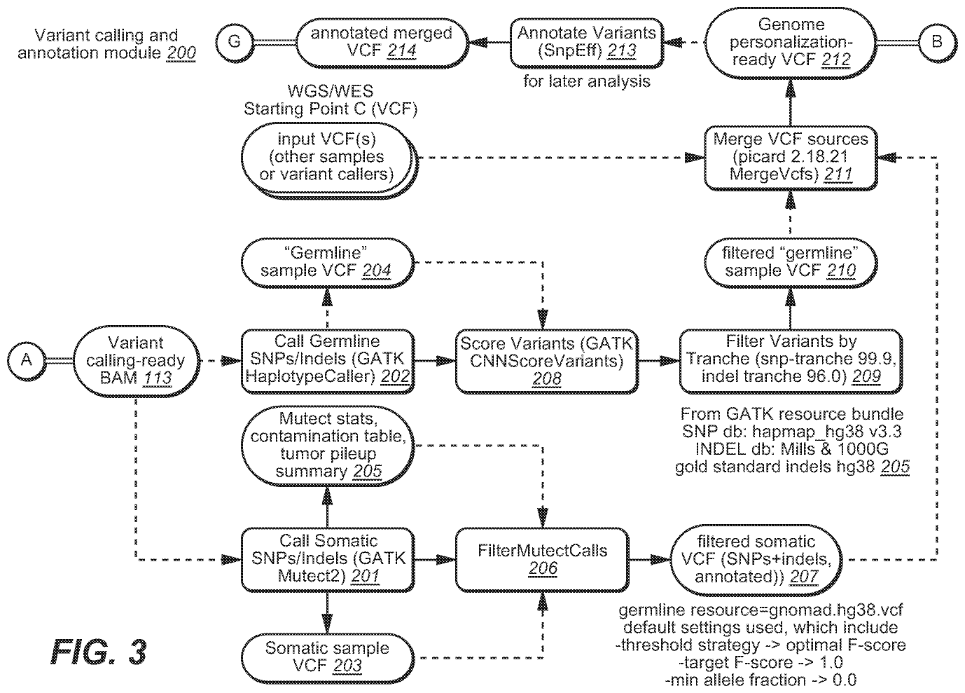

FIG. 3 illustrates the operations of variant calling module 200, according to one embodiment of the present invention. As shown in FIG. 3, BAM files 113 represent both output files 31 and 32 (from circle “A” in FIG. 2)—containing, respectively, aligned and recalibrated reads of the MN DNA sequences and aligned and recalibrated reads of the target DNA sequences. Output files 31 and 32 are provided to somatic variant caller 201 and genotyping variant caller 202, respectively. Somatic variant caller 201 may be implemented, for example, by the Mutect2 tool in GATK and genotyping variant caller 202 may be implemented, for example, by HaplotypeCaller in GATK. In this embodiment, Mutect2 and HaplotypeCaller only call variants related to SNPs and indels. The variants called that correspond to somatic alternative alleles are provided in somatic sample VCF file 203, and the variants called that correspond to germline alternative alleles are provided in germline sample VCF file 204.

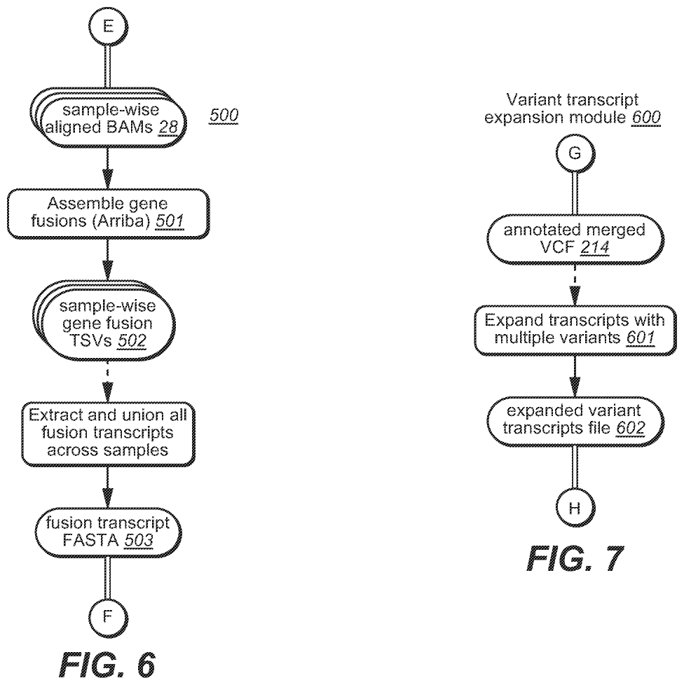

In addition to somatic sample VCF file 203, Mutect2 provides quality data file 205 (e.g., read statistics, contamination tables, and tumor pileup summary) Somatic sample VCF file 203 may be filtered at step 206 according to quality data file 205 using, for example, the FilterMutectCalls tool in GATK, which provides filtered somatic sample VCF file 207. In this detailed description, the variants in filtered somatic sample VCF file 207 are referred to as “somatic variants.” In this embodiment, a deep-learning model, referred to as “convolutional neural network” (CNN), or another suitable machine learning platform, may be used at step 208 to score the germline alternative alleles in germline sample VCF 204. Scoring may be carried out using, for example, CNNScoreVariants in GATK at step 207. The scores allow filtering of germline sample VCF based on quality percentiles (e.g., 99.9 for the SNP tranche and 96.0 for the indel tranche), or any suitable figure of merit, at step 209 to provide filtered germline sample VCF 210. In this detailed description, the variants in filtered germline sample VCF file 210 are referred to as “germline variants.” Filtered germline sample VCF 210 and filtered somatic sample VCF 207 may be merged at step 211 using, for example, the MergeVCFs tool in Picard to provide customization-ready genome VCF file 212. (Customization-ready genome VCF file 212 corresponds to called variants file 24 of FIG. 1.) FIG. 3 indicates customization-ready genome VCF file 212 being available for further processing by circle “B.” As shown in FIG. 3, at step 213, customization-ready genome VCF file 212 may be annotated to provide annotated merged genomic VCF file 214 using, for example, the SnpEff tool, which is also commercially available as ClinEff at dnaminer.com. SnpEff modifies customization-ready genome VCF file 212 in accordance to a reference genome annotation (e.g., the one provided by the public research consortium GENCODE) to append, onto each variant, known transcript identifier, codon position and other information. As in indicated by circle “G,” annotated merged genomic VCF file 214 may be provided to variant transcript expansion module 600 (FIG. 7) for further processing.

Although variant calling in this embodiment of the present invention is illustrated herein using Mutect2 and HaplotypeCaller, which are capable only of short-length mutations (e.g., SNPs, indels and relatively short-length MNPs), the present invention is not limited thereby. A variant caller capable of detecting larger structural variants (e.g., gene fusion or tandem duplications) may also be used. An example of a variant caller capable of detecting tandem duplication is Pindel, developed at the Wellcome Sanger Institute.

### Customized Diploid Genome Module 300

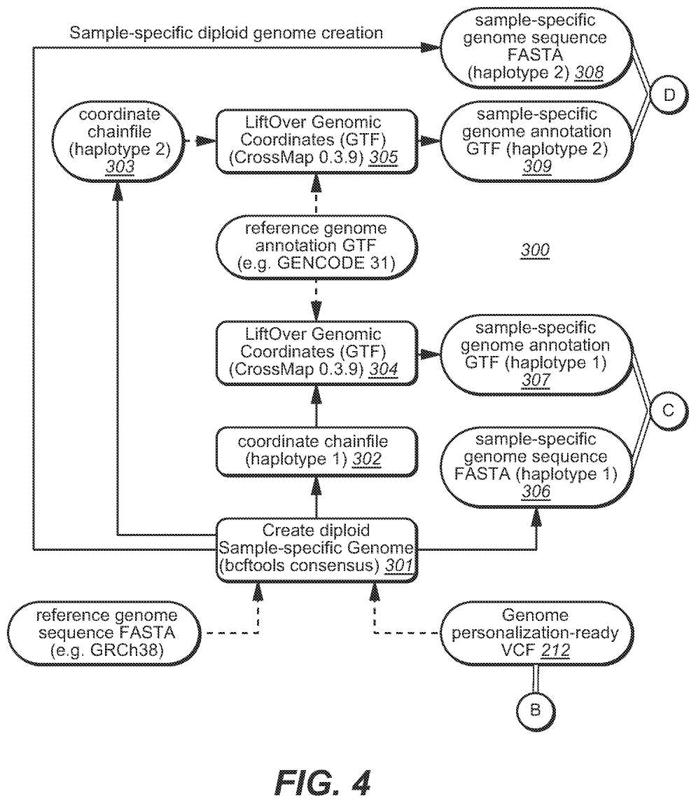

Customized diploid genome module 300 creates the customized genome based on customization-ready genome VCF file 212 (corresponding to called variants file 24 of FIG. 1). FIG. 4 illustrates the operations of customized diploid genome module 300, according to one embodiment of the present invention. In this embodiment, the customized genome is provided in first and second Haplotype files 306 and 308 (i.e., corresponding to file sets 25 and 26 of FIG. 1, respectively) using diploid genome assembly tool 301. Diploid genome assembly tool 301 may be implemented, for example, by the Consensus tool in bcftools. Consensus modifies the reference genome by incorporating the germline and somatic variants parsed from customization-ready genome VCF file 212 (circle “B”; see, corresponding circle “B” in FIG. 2). In this operation, Consensus incorporates homozygous germline alternative alleles in both first and second Haplotype files 306 and 308 (i.e., corresponding to file sets 25 and 26 of FIG. 1, respectively), while somatic alternative alleles are incorporated into second Haplotype files 308 (i.e., file set 26). Consensus creates first and second Haplotype files 306 and 308 in the FASTA format.

Up to this point, all reads have been aligned relative to the reference genome. After incorporation of the germline and somatic variants in customization-ready genome VCF file 212, genomic coordinates in the annotations need to be adjusted to the genomic coordinates of the customized genome. This adjustment is enabled by “chain” files 302 and 303 provided by Consensus. The genomic coordinates in the annotations of first and second Haplotypes are adjusted and updated at steps 304 and 305 to provide annotation files 307 and 309 for first and second Haplotype files 306 and 308, respectively. A suitable tool for this “lift over” is provided by the CrossMap tool known to those of ordinary skill in that art. All reads in first and second haplotype files 306 and 308 are now aligned relative to the concurrently created customized genome. Circles “C” and “D” indicate (i) First Haplotype file 306 and its accompanying annotation file 307 (“First Haplotype file set”), (ii) second Haplotype file 308 and its accompanying annotation file 309 (“Second Haplotype file set”), respectively.

### Customized Transcriptome Assembly Module 400

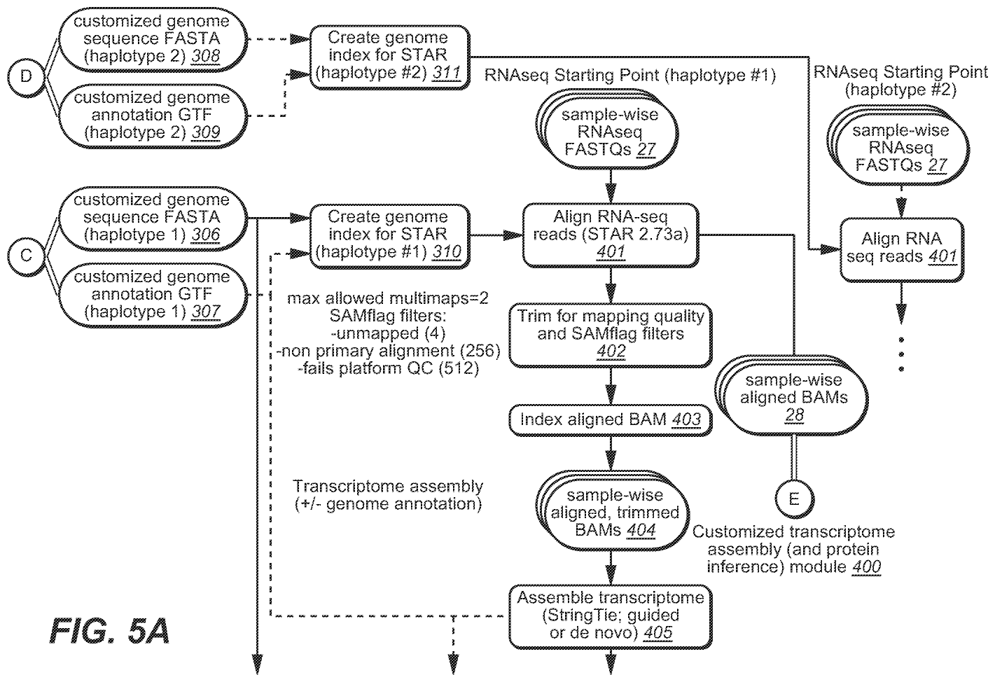

The present invention predicts a customized transcriptome that is assembled based on the customized genome and from RNA sequencing data. FIG. 5 illustrates the operations of customized transcriptome assembly module 400, according to one embodiment of the present invention. In this embodiment, the RNA sequencing data in First Haplotype file set and Second Haplotype file set are expected to be aligned to the customized genome using the STAR (“Spliced Transcript Alignment to a Reference”) tool, known to those of ordinary skill in the art. As indicated by circles C and D respectively indicate First and Second Haplotype file sets obtained in FIG. 3. As the alignment step in STAR utilizes its proprietary index to its reference genome, first and second Haplotype files 306 and 308, in First and Second Haplotype file sets are indexed using STAR at steps 310 and 311, respectively, to provide corresponding STAR-indexed Haplotype file sets, as shown in FIG. 4.

In addition to the customized genome, customized transcriptome assembly module 400 receives as input one or more RNA sequencing files 27. Each of RNA sequencing files 27 may include a separate tissue sample (e.g., belonging to a separate read group). For the reads in each RNA sequence file, customized transcriptome assembly module 400 (i) aligns and indexes the reads from RNA sequencing file 27 separately to each Haplotype file set in the customized genome (i.e., reads in RNA sequencing files 27 are separately aligned to first and second Haplotype files 306 and 308), (ii) assembles a customized transcriptome for each Haplotype file set, (iii) creates a customized predicted proteome for each Haplotype, based on the corresponding transcriptome, to provide a predicted proteome for the Haplotype; and (iv) merging the predicted proteomes to form the customized proteome.

In this embodiment, RNA sequencing data files 27 are each aligned at step 401 using, for example, the STAR tool, which provides customized aligned BAM files 28. Customized aligned BAM files, aligned to the custom genome, may be used for suitable further processing (indicated by circle “E”). For example, in the application of FIG. 1, a set of BAM files 28 are prepared by STAR from each of STAR-indexed Haplotype files 306 and 308. BAM files 28 may also be augmented with additional cDNA nucleotide sequences referenced to the customized genome using a suitable tool that suggests possible structural genomic rearrangements. As a messenger RNA sequence may be transcribed from multiple exons in the customized genome, each read may have sections at different alignments in the customized genome. At step 402, the set of aligned reads is trimmed for quality (e.g., discarding reads with poor mapping scores or those that have been flagged by the sequencer as low quality). Chimeric reads or alignments are retained and, for each read, the alignment with the highest mapping score is annotated “primary alignment.” A large number (e.g., 256) of non-primary alignments may be retained. At step 402, the trimmed set of reads may be filtered for various quality improvements using, for example, the SAMflags tool in SAMtools. The filtered reads may then be indexed according to genomic coordinates at step 403 using, for example, the BuildBAMIndex tool in Picard. Thus, for each Haplotype, customized aligned BAM files 404 each correspond to reads of a different tissue sample or read group.

At step 405, customized aligned BAM files 404 are each assembled into a detected transcriptome annotation using, for example, StringTie in either a guided mode, or is assembled de novo. StringTie is a transcript assembly and quantification tool that is available from the Center for Computational Biology at the Johns Hopkins University. In this embodiment, a transcriptome annotation comprises transcripts that are represented by lists of exon (coding region) boundaries denoted by genomic coordinates. At step 406, each of the detected transcriptome annotation files (one per BAM file 404) for each Haplotype are merged in a non-redundant manner into a merged transcriptome annotation file 407 using, for example, Merge in StringTie. At this point, there is one merged transcriptome file per Haplotype.

At step 408, based on their alignments, the complementary DNA (cDNA) nucleotide sequences of all the transcripts or transcript fragments in the detected transcriptome annotation are read out from the customized genome and stored in detected transcriptome sequence file 409. Transcriptome sequence file 409 may be provided as libraries in FASTA format.

Recalling that, in this embodiment, variant calling and annotation module 200 does not call variants beyond SNP, short indels and short MNPs, other structural rearrangements may be detected by other means. In this embodiment, additional cDNA nucleotide sequences referenced to the customized genome may be added to detected transcriptome sequence file 409 at step 410. Such additional cDNA nucleotide sequences for each Haplotype may be provided, for example, from a tool that detect other structural rearrangements (e.g., gene fusion module 400), as indicated by circle “F.” The additional cDNA nucleotide sequences may be included, for example, in output fusion transcription PASTA file 503. Similarly, supplementary nucleotide sequences deriving from the variant transcript expansion module 600, which account for variants in close chromosomal proximity whose cis vs trans strand phasing is not ready resolvable, may also be appended at this at step 410.

The nucleotide sequences in transcriptome sequence file 409 are then translated into amino acid sequences at step 411 to identify—from the customized genome—candidate open reading frames (ORFs) using, for example, the Transdecoder.LongORFs tool in Transdecoder. An open reading frame or ORF is a continuous sequence of codons, beginning with a start codon and ending with a stop codon. (A codon is a three-nucleotide sequence that typically maps to an amino acid,) In this embodiment, a candidate ORF has at least 70 codons (i.e., 210 nucleotides). It is not uncommon that one or more candidate ORFs may be inferred from a single transcript. Transdecoder is open-source software, but commercial versions are available (e.g., from Biobam Bioinformatics, Cambridge, Mass.).

Transcriptome sequence file 409 is also used, at step 412, to query a protein database (e.g., UniProt, available from uniprot.org) for homologous sequences using a sequence search tool (e.g., BLASTp, available from the National Center for Biotechnology Information (NCBI)). Based on the candidate proteins returned from the protein database and the candidate ORFs identified at step 411, the ORFs that have the highest likelihood of being translated into functional proteins (i.e., collectively, customized predicted transcriptome for the Haplotype) are predicted using, for example, the TransDecode.Predict tool, which combines the homology information ascertained by sequence search with a Markov-based machine learning scoring model. At step 413, proteins in the customized predicted transcriptome for the Haplotype are mapped to genomic coordinates of the customized genome, using a remap tool in TransDecoder, for example. In this manner, a customized Haplotype-specific proteome 419 is assembled for each Haplotype. In one embodiment, the customized predicted Haplotype-specific proteome file 419 may be provided in FASTA format, with an accompanying BED format file that maps the predicted proteins to their respective chromosomal loci in the customized genome.

The customized Haplotype-specific predicted proteomes 419, prepared from first and second Haplotype files 306 and 308, respectively, are merged to form customized predicted proteome 420.

### Gene Fusion Module 500

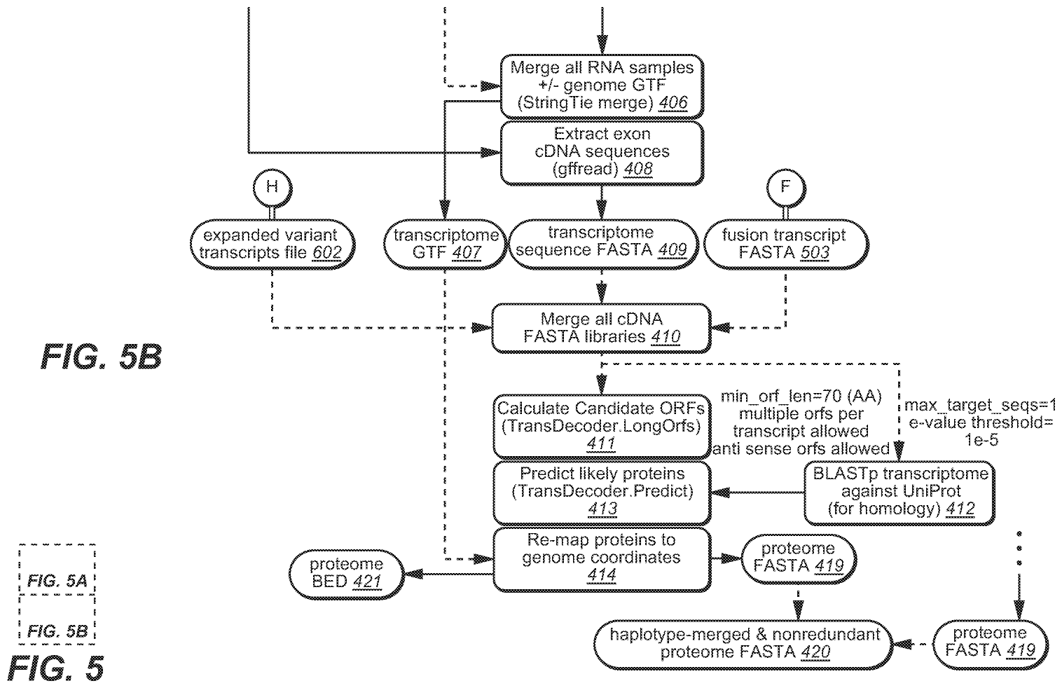

FIG. 6 illustrates the operations of gene fusion module 500, according to one embodiment of the present invention. As discussed above in conjunction with FIG. 5, alignment at step 401 by STAR provides aligned RNA sequencing files 28 for each Haplotype. Aligned RNA sequencing files 28 for each Haplotype are then received into gene fusion module 500 for analysis, as shown at step 501. Analysis at step 501 may be, for example, detecting evidence of possible structural genomic rearrangements (e.g., gene fusion, and tandem and other duplications) using the Ariba tool, which may be obtained from the German Cancer Research Center DKFZ, Applied Bioinformatics, Heidelberg, Germany. For each tissue sample, Arriba assembles transcripts that exhibit such structural rearrangements (i.e., possible gene fusion transcript files 502) in TSV format files gene fusion TSV files 502). The transcripts in gene-fusion transcript files 502 for the various tissue samples are merged in a non-redundant manner to provide gene fusion transcriptome file 503. Gene fusion transcriptome file 503 may be provided for any suitable further processing (circle “F”). For example, gene fusion transcriptome file 503 may be appended to detected transcriptome sequence file 409 at step 410, as discussed above (see, circle “F” in FIG. 4).

### Variant Transcript Expansion Module 600

FIG. 7 illustrates the operations of variant transcript expansion module 600, according to one embodiment of the present invention. As discussed above in conjunction with FIG. 3, variant calling and annotation module 200 detects germline and somatic variants in a whole genome or exome dataset and yields merged VCF file 212 that is suitable for using as input for genome customization. At step 601, annotated merged genome VCF file 214 (indicated by circle “G,” corresponding to circle “C” in FIG. 3) is then provided to variant transcript expansion module 600 for analysis. Analysis at step 601 includes, for example, parsing merged genome VCF file 214 for variants that reside on a shared transcript and whose codon position are within a certain distance of each other (e.g., a 25-codon sequence, which is a length of frequently used typical tryptic peptides in proteomic analysis). In this embodiment, transcripts containing two or more variants satisfying the afore-mentioned criteria are “expanded” by a factor of 2^{N}, where N is the number of variant loci satisfying the criteria, such that there are subsequently additional “copies” of the given transcript sequence comprising every possible combination of alleles (reference or variant) that may occupy each of the variant loci. Resulting expanded variant transcripts file 602 may, be appended to detected transcriptome sequence file 409 at step 410, as discussed above (circle “H”; see, corresponding circle “H” in FIG. 4).

### Post-Processing

Based on annotated genomic VCF file 213, customized predicted proteome 420, and a protein database (e.g., UniProt), one may predict (i) the homologous proteins found in the protein database that may result from the transcripts that incorporate the called germline and somatic variants in customized genome VCF file 212, and (ii) the peptides that may be detected by mass spectrometry after cleaving such homologous proteins with appropriate enzymes at one or more selected amino acid residues, as known to those of ordinary skill in the art. Customized genomic VCF file 213 allows relating the predicted changes in amino acid sequences to the mutations that give rise to the germline and somatic variants.

Protein extract from actual user tissue samples may be broken down and analyzed by mass spectrometry qualitatively and quantitatively using, for example, the MaxQuant tool, available from Max Planck Institute of Biochemistry. In this example, the MaxQuant tool is guided by the search space defined by customized predicted proteome 420. Detection of any peptide predicted in customized predicted proteome 420 helps identify the genes in the customized genome that are actually transcribed and translated.

The above detailed description is provided to illustrate specific embodiments of the present invention and is not intended to be limiting. Numerous modifications and variations within the scope of the invention are possible. The present invention is set forth in the following accompanying claims.

We claim:

1. A process for predicting a proteome based on one or more tissue samples of an individual, comprising:
identifying somatic and germline variants based on a reference genome and nucleotide sequences derived from the tissue samples;
constructing a customized genome by modifying the reference genome based on the somatic and germline variants identified;
aligning RNA sequences derived from the tissue samples to transcription loci in the customized genome;
assembling a detected transcriptome with transcripts derived from the aligned RNA sequences; and
associating the detected transcriptome with proteins in a protein database and including the associated proteins in the proteome.

2. The process of claim 1, wherein the tissue samples comprise a tissue sample obtained from a diseased site (“target sample”) and a matched normal or virtual normal tissue sample.

3. The process of claim 2, wherein the somatic variants comprise alternative alleles found in target sample, relative to alleles in the matched normal or virtual normal tissue sample.

4. The process of claim 2, wherein the germline variants comprise alternative alleles found in either the target sample or the matched normal or virtual normal sample, relative to alleles in the reference genome.

5. The process of claim 1, wherein the nucleotide sequences are provided from a whole genome sequencing (WGS) or whole exome sequencing (WES) procedure.

6. The process of claim 5, further comprising unmapping the nucleotide sequences from the WGS or WES procedure.

7. The process of claim 1, wherein the somatic and germline variants include structural rearrangement variants other than single-nucleotide polymorphisms and single-nucleotide insertion or deletion mutations.

8. The process of claim 1, wherein the identified germline variants are assessed for quality using a deep-learning model.

9. The process of claim 8, wherein the deep-learning model is implemented on a convolutional neural network.

10. The process of claim 1, wherein the customized genome comprises a first group and a second group, wherein the first group includes (i) the germline variants and (ii) homozygous somatic variants, and wherein the second group includes the somatic variants.

11. The process of claim 10, wherein a partial detected transcriptome is assembled for each of the first and second groups and wherein the detected transcriptome is formed by merging the partial detected transcriptome.

12. The process of claim 1, further comprising detecting in the aligned RNA sequences transcripts that correspond to structural rearrangements in the customized genome.

13. The process of claim 12, wherein the structural rearrangements comprise one or more of: gene fusion and tandem or exon duplications.

14. The process of claim 12, further comprising including the detected transcripts that correspond to structural rearrangements in the customized genome in the detected transcriptome.

15. The process of claim 1, further comprising extracting exons from transcripts in the assembled detected transcriptome and using the extracted exons to identify open read frames in the customized genome.

16. The process of claim 15, further comprising identifying proteins in the protein database corresponding to the identified open read frames.

17. A bioinformatics system configurable and operable on one or more processors, comprising:
a variant calling module configured to identify somatic and germline variants based on a reference genome and nucleotide sequences derived from the tissue samples;
a customized genome module configurable to construct a customized genome based on modifying the reference genome according to the somatic and germline variants identified; and
a customized transcriptome assembly module configurable to: (i) align RNA sequences derived from the tissue samples to transcription loci in the customized genome; (ii) assemble a detected transcriptome with transcripts derived from the aligned RNA sequences; (iii) associate the detected transcriptome with proteins in a protein database; and (iv) include the associated proteins in the proteome.

18. The bioinformatics system of claim 17, wherein the one or more processors accessible by a user of the bioinformatics system over a wide area computer network,

19. The bioinformatics system of claim 18, wherein the one or more processors comprise graphics processor units.

20. The bioinformatics system of claim 17, wherein the tissue samples comprise a tissue sample obtained from a diseased site (“target sample”) and a matched normal or virtual normal tissue sample.

21. The bioinformatics system of claim 20, wherein the somatic variants comprise alternative alleles found in target sample, relative to alleles in the matched normal or virtual normal tissue sample.

22. The bioinformatics system of claim 20, wherein the germline variants comprise alternative alleles found in either the target sample or the matched normal or virtual normal sample, relative to alleles in the reference genome.

23. The bioinformatics system of claim 17, wherein the nucleotide sequences are provided from a whole genome sequencing (WGS) or whole exome sequencing (WES) procedure.

24. The bioinformatics system of claim 23, further comprising an alignment module configurable to align the nucleotide sequences from the WGS or WES procedure to a reference genome.

25. The bioinformatics system of claim 17, wherein the variant calling module calls somatic and germline variants with structural rearrangements other than single-nucleotide polymorphisms and single-nucleotide insertion or deletion mutations.

26. The bioinformatics system of claim 17, wherein the germline variants are assessed for quality using a deep-learning model.

27. The bioinformatics system of claim 26, wherein the deep-learning model is implemented on a convolutional neural network configured on the one or more processors.

28. The bioinformatics system of claim 17, wherein the customized genome comprises a first group and a second group, wherein the first group includes (i) the germline variants and (ii) homozygous somatic variants, and wherein the second group includes the somatic variants.

29. The bioinformatics system of claim 28, wherein a partial detected transcriptome is assembled for each of the first and second groups and wherein the detected transcriptome is formed by merging the partial detected transcriptome.

30. The bioinformatics system of claim 17, further comprising a gene fusion module configurable to detect in the aligned RNA sequences transcripts that correspond to structural rearrangements in the customized genome.

31. The bioinformatics system of claim 30, wherein the structural rearrangements comprise one or more of: gene fusion and tandem or exon duplications.

32. The bioinformatics system of claim 30, further comprising including the detected transcripts from the gene fusion module in the customized genome in the detected transcriptome.

33. The bioinformatics system of claim 17, further comprising extracting exons from transcripts in the assembled detected transcriptome and using the extracted exons to identify open read frames in the customized genome.

34. The bioinformatics system of claim 33, further comprising identifying proteins in the protein database corresponding to the identified open read frames.

35. The process of claim 1, wherein when multiple variant loci are included in a peptide fragment of a length within a predetermined range, the somatic and germline variants include more than one possible combination of including one or more of the multiple variant loci.

36. The process of claim 35, wherein the predetermined range spans 5 to 30 nucleotides, inclusive.

37. The bioinformatics system of claim 17, wherein when multiple variant loci are included in a peptide fragment of a length within a predetermined range, the somatic and germline variants include more than one possible combination of including one or more of the multiple variant loci.

38. The bioinformatics system of claim 37, wherein the predetermined range spans 5 to 30 nucleotides, inclusive.
